(19) United States
(12) Patent Application Publication
(10) Pub. No.: US 2005/0153293 A1
(43) Pub. Date: Jul. 14, 2005

(54) Nucleic acid analysis

(76) Inventor: John Douglas Oultram, Stretford, Manchester (GB)

Correspondence Address: WOODARD, EMHARDT, MORIARTY, MCNETT & HENRY LLP, BANK ONE CENTER/TOWER, 111 MONUMENT CIRCLE, SUITE 3700, INDIANAPOLIS, IN, 46204-5137, US

(21) Appl. No.: 10/503,386

(22) PCT Filed: Feb. 3, 2003
(86) PCT No.: PCT/GB03/00455

(30) Foreign Application Priority Data
Feb. 4, 2002 (GB) 0202462.8

(51) Int. Cl. C12Q 1/68; C07H 21/04; C12P 19/34

(52) U.S. Cl. 435/6; 435/91.2; 536/24.3

(57) ABSTRACT

A method of analysing a nucleic acid sequence comprises digesting the sequence with a restriction enzyme which cleaves the nucleic acid to produce fragments with overhangs containing at least one partially random or at least one semi-random base. The fragment mixture may be analysed to determine the relative size of the fragments and the sequences of their ends. Alternatively the fragment mixture may be analysed to determine the identity of at least one of said random or semi-random bases and optionally to determine the relative size of the fragments to obtain sequence data that can be used to order the fragments relative to each other to generate partial or complete restriction maps of said nucleic acid sequence.

### FIELD OF INVENTION

The present invention relates to the analysis of nucleic acids, e.g. to determine a restriction map thereof.

### BACKGROUND OF THE INVENTION

The mapping of restriction sites on nucleotide sequences is an important tool in molecular biology and is a fundamental starting point for many areas of study including large scale sequencing projects, such as the human genome project, as well as comparative genetics, operon research etc.

When presented with a large piece of DNA of unknown sequence, from which genetic information is required, a usual first step is to map the sequence with restriction endonuclease enzymes (REN). This is done by digesting the unknown nucleotide sequence with a number of restriction enzymes, either singly or in concert. The fragments that are produced by digestion of the unknown sequence are then separated, usually by electrophoresis through an agarose gel, visualised by staining, photographed and their sizes ascertained by comparison with a ‘ladder’ of fragments of known size. Knowing the sizes of fragments generated by enzymes A and B, for instance, together with the sizes of fragments generated by enzymes A and B in concert, allows the sites for restriction enzymes A and B to be placed relative to each other generating a rudimentary restriction map of the unknown DNA double strand. These maps may contain logical uncertainties in which a number of potential fragment orders will fit the data generated by restriction digestion. These are usually resolved by mapping with further combinations of restriction enzymes such that the final product is an unequivocal map of the sequence in question. In order to produce fine detail restriction maps it is often necessary to use restriction enzymes whose recognition sites involve a short number of base pairs, usually four, in conjunction with enzymes whose sites are longer, e.g. six base pairs. (The recognition sequence is that sequence that must be present for the enzyme to recognise and cleave the site). This is because a particular sequence of n bases will occur on average every 4ⁿ bases in a random sequence of DNA. As an example, cut sites for a restriction enzyme with a four base recognition sequence will occur statistically more often (i.e. every 256 bp) than sites for an enzyme with a six base sequence (i.e. every 4,096 bp).

Restriction maps are used to for a number of purposes. These include using the map to orient and position the fragment within a larger DNA strand by comparing maps of sub-fragments of the larger DNA strand for areas of overlap. The map can also be used to inform sub-cloning strategies such that clones can be created whose position is known with respect to other sub-clones on the map.

Inconsistencies in a restriction map arise because all of the sites of restriction are usually identical or near identical and information is not available about the nature of any particular site. Thus these sites can be arranged in any order and the function of restriction in concert with one or more other RENs is unequivocally to order the fragments by reference to both. Thus there are disadvantages to the restriction mapping techniques currently used in the art in that the techniques can be long winded, requiring the use of many restriction enzymes to generate a suitable map.

It is an object of the present invention to obviate or mitigate the above mentioned disadvantages.

### SUMMARY OF THE INVENTION

According to a first aspect of the present invention there is provided a method of analysing a nucleic acid sequence comprising
(i) digesting the sequence with a restriction enzyme which cleaves the nucleic acid to produce fragments with overhangs containing at least one partially random or at least one semi-random base; and
(ii) analysing the fragment mixture obtained from (i) to determine the relative size of the fragments and the sequences of their ends.

According to a second aspect of the present invention there is provided a method of analysing a nucleic acid sequence comprising
(i) digesting the sequence with a restriction enzyme which cleaves the nucleic acid to produce fragments with overhangs containing at least one partially random or at least one semi-random base;
(ii) analysing the fragment mixture obtained from (i) to determine the identity of at least one of said random or semi-random bases and optionally to determine the relative size of the fragments.; and
(iii) using the sequence data derived from (ii) to order the fragments relative to each other to generate partial or complete restriction maps of said nucleic acid sequence.

By random we mean that one or more bases at a given position or positions in the overhang will be any possible base of the type present in the nucleic acid sequence. Thus, for example, in the case of DNA the overhang would randomly include A, G, T or C at a given position. By semi-random we mean that at a given position or positions in the overhang there will be a base which will not be uniquely known but will not be random in the same sense as described above. Thus, for example, in the case of DNA a semi-random base would be one of three or more preferably two possibilities.

We have found that determining the sequences of the overhangs in fragments of a restriction digest of a nucleic acid using an enzyme that provides overhangs containing random and/or semi-random bases provides a convenient way of mapping an unknown nucleic acid sequence at high resolution and yet with sufficient base specificity to allow ready manipulation using molecular biology techniques (e.g. subcloning).

The invention may be used for the analysis of DNA and may be applied to genomic DNA from any source, clones or sub-clones derived from such sources or cDNA derived from an original mRNA sample. The invention may also be applied to other forms of nucleic acid, e.g. Protein Nucleic Acid (PNA), Locked Nucleic Acid (LNA).

In the method of the invention the unknown nucleic acid strand is restricted with a restriction enzyme whose recognition site can be characterised as possessing the following properties:
(i) the restriction site recognition sequence must be of sufficient complexity to cut a random nucleic acid (e.g. DNA) sequence sufficiently often for the mapping resolution of the task for which the map is being prepared. For most mapping purposes this will essentially be as often as possible and thus a four or five base recognition sequence would be preferable.
(ii) upon restriction the cleavage site must contain random or semi-random nucleotide sequence of sufficient overhang to allow the specific site to be characterised to some extent and differentiated from other cleavage sites for the enzyme.

The specific restriction sites are differentiated by a sequence specific means and the different specific cleavage sites are then used to generate mapping information on the DNA strand from which the fragments arose.

The differentiating means could be:

Detection of probes whose sequence in whole or in part is specifically complementary to one or more overhang sequences. For instance, a fluorophore or radiolabel might be used to ‘tag’ a specific sequence or group of sequences and then these could be detected by hybridisation or by hybridisation followed by ligation onto the fragments on the basis that the hybridisation reaction will be sequence specific with regard to the group of overhang sequences.

PCR amplification of specific sub-groups of the fragment population dependent upon the sequences of the primers involved. For example if the overhang created by enzyme digestion contained four random bases a specific subset of PCR primers could be synthesised containing all combinations of three of the bases but containing only a specific base at the fourth position. If this primer set were ligated to the restriction fragments and used to amplify them only those fragments which had the base complementary to the primer at the fourth base position would amplify and this would identify those fragments for mapping and sub-cloning purposes.

The method of the invention does not disclude separating the steps of interrogating the fragments for sequence information and for length information. It is known in the art that oligonucleotides may be attached to solid phases such as beads or slides and that these oligonucleotides can be made to capture their complementary strands in a sequence specific manner. It is further known that the oligonucleotides may be specially separated to form an array whose elements each have a known sequence. It could be envisaged that an array of probes of or containing sequences that would capture restriction fragments in a sequence specific manner might be arrayed onto a solid phase as described and that a target nucleic acid might be digested by a restriction endonuclease of the type outlined and the digest applied to a solid phase where the capture of subfragments could be detected to give sequence data about the end of the captured fragment without necessarily disclosing any information as to the size of the fragment. In order to generate a restriction map from this information it would be necessary to identify the overhang sequences on each end of a fragment i.e. to link together the individual overhanging sequences determined by the first array method. This may also be performed without generating data on fragment size by such methods as recovering the fragments annealed to each element of the array separately and applying this to a second array such that the overhang at the unannealed end of the fragment might anneal to its complement thus disclosing the identity of both overhangs. The sequence data thus generated might be used according to the method of the invention to generate a topological restriction map in which the order of restriction sites was known but not the sizes of the fragments produced. Alternatively, both size and linkage data might then be generated together and independently of the sequence data from the first array by for instance releasing the captured fragments from each array element in turn and determining the size of the released fragment by methods known in the art including gel electrophoresis. Since each fragment should anneal at two sites, complementary to its two ends, identically sized fragments should be obtained from two elements of the array disclosing the sequence of the ends of the fragment. Fragments that have identical sequences at their ends will anneal to only single elements of the array and will appear as orphan fragments i.e. with no partner from a different element of the array and may be identified thus.

It is well known that techniques reliant upon hybridisation specificity may be subject to experimental ‘background noise’ due to inexact hybridisation of fragments largely but not entirely complementary to the target. It is a disclosure of the method of the invention that and optional additional enzyme reactions may be carried out after hybridisation or where appropriate after hybridisation and ligation in order to eliminate the false signal generated by such mismatch hybridisation. Such an enzyme reaction may optionally be used in order to specifically degrade mismatch regions while not effecting fully complementary matches. Suitable enzymes for use in this optional additional step include Mung Bean Nuclease and T7 endonuclease (New England Biolabs catalogue 2000-2001, www.neb.com).

Another optional treatment which may reduce background noise might be treatment with an enzyme which would degrade unincorporated labelled primers or probes thus removing interfering background from analyses such as gel electrophoresis. A suitable enzyme for this treatment might be Escherichia coli endonuclease I (New England Biolabs).

### DESCRIPTION OF PREFERRED EMBODIMENTS

Preferably the restriction enzyme produces overhangs of 3 or more bases, and more preferably 6 or more, e.g. 7 to 10, bases. Preferably also at least 3 (e.g. 4) of the bases in the overhang are random. At least some of the random bases may be at the “free” end of the overhang. Alternatively or additionally at least some of the random bases may be at the end of the overhang that is attached to the main sequence. Preferably also the overhang contains at least one semi-random base which is one of two possibilities.

A suitable restriction enzyme for use in analysis of DNA is TspRI. TspRI cuts the following site on double stranded DNA;

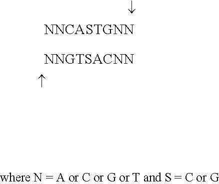

Thus in the above formula N represents a random base in the context of the present invention whereas S represents a semi-random base.

The overhangs may be 3′ or 5′ overhangs however a further clear advantage is gained in the use of restriction enzymes that cleave to leave long (6 or longer) 3′ overhangs such as TspRI and the advantage that the use of an enzyme with a 3′ overhang of longer than 6 bases whose sequence can be wholly or largely deduced confers is a particular disclosure of the invention. The advantage lies in the fact that upon construction of a map and determination of cleavage sequences any fragment or contiguous sequence containing a number of cleavage sites may be amplified from the background nucleic acid by amplification methods known in the art using primers complementary to the fragment overhangs or having such complementary sequences at their 3′ ends.

An advantageous embodiment of such amplification procedure is disclosed. Such embodiment involves totally or partially digesting the target nucleic acid with the restriction enzyme and then inactivating it if necessary and then hybridising to the fragment mix primers whose sequence or whose 3′ end sequence is complementary to the ends of the fragment to be amplified. The primers may then be further optionally ligated to the ends of the fragment. The mixture is then treated with a DNA polymerase which will generate a copy of the target fragment from the primers without the step of separating the strands of double stranded target nucleic acid which would ordinarily be necessary for the first round of a conventional amplification reaction. The sample is then treated as for any amplification process known in the art, such as the polymerase chain reaction (PCR).

The particular advantage disclosed in this method is that the treatment of the mixture before any denaturing step has been performed vastly reduces the amount of target sequence which is single stranded at this critical early stage and might interfere with the reaction or generate false priming events giving rise to spurious results. This is of particular importance when the overhangs are shorter than conventional amplification primers (i.e. less than 15 or so bases) as hybridisation of short primers must be carried out at lower stringency than for longer primers and the risk of mispriming increases with the lowering of stringency. It is also possible to use amplification strategies other than PCR wherein the not hybridised DNA fragments would remain largely double stranded throughout the entire amplification process. Such strategies might include Strand Displacement Amplification (U.S. Pat. No. 5,455,166 (Becton Dickinson Co. et al)) or Isothermal solution phase amplification (WO-A-9909211 (Tepnel Medical Ltd et al).

An important part of the invention is determining the sequences of the overhangs in the fragments of the restriction enzyme digest. This may be done in a number of ways. In general, these will involve the use of libraries of probes constructed on the basis of the information known about the overhangs.

Thus consider, for example, a restriction digest comprised of fragments having, at each end, overhangs with
(i) known bases at known positions;
(ii) a semi-random base (chosen form one of two possibilities) at a known position; and
(iii) random bases at known positions.

To determine the identity of the semi-random base in each overhang of each fragment then the following procedure may be adopted. Two libraries of probes are constructed. Each library is comprised of probes having a sequence for potential hybridisation to the overhangs. In one library the probes have, at a position corresponding to the semi-random base, one of the two possibilities therefor. The other library comprises probes with the other possibility. Additionally the probes of each library are such that they incorporate bases that will hybridise to the known bases (i) in the overhang and for other positions in the sequence comprise all possible combinations of bases.

The two libraries may be used separately of each other and also together to “interrogate” separate samples of the digest. This procedure will involve the use of hybridising conditions so that probes in the library that are fully complementary to the overhangs will hybridise thereto. It may also be desirable to ligate the hybridised probes. Optionally, a further enzymatic digestion may be performed to degrade those sequences which are not wholly complementary to their target. Suitable enzymes for use in this optional additional step include Mung Bean Nuclease and T7 endonuclease. A determination is made as to whether probes for any one library have hybridised to none, one or both ends of each fragment.

For the purposes of detection the probes may be fluorescently labelled. Alternatively subsequent to hybridisation an amplification reaction may be conducted using the primer library with which the sample was originally interrogated.

All of these procedures are described more fully below for use of the enzyme TspR1.

Analysis of the product mixture (e.g. by separation on agarose gel) allows not only the relative sizes of the fragments to be identified but also for each size fragment whether it is heterospecific (i.e. it has a different semi-random base in its two overhangs) or homospecific (i.e. it has the same two semi-random bases in its overhangs).

It will be appreciated that in the case where the overhangs contain two or more semi-random bases then an appropriate number of primer libraries may be constructed so as to identify which semi-random base combination is present in each overhang. Thus, for example, if the overhang has two semi-random bases then 4 primer libraries will be constructed, one with its two semi-random bases being the same and being selected from one possibility, one with its semi-random bases being the same but selected from the other possibility, one with its semi-random bases being different but in a particular order and the other with its two semi-random bases being different but in the other order.

It is then possible to identify each random base in the two overhangs of a fragment as being one of two possibilities, and in some cases it is possible uniquely to identify the base. Consider for example that the overhang has n random bases along with the known bases and a (single) semi-random base. Two primer libraries may be constructed each with n “families” of primers. Each “family” of a particular library includes probes with sequences comprising
(a) bases complementary to the known bases of the overhang at their known positions;
(b) a semi-random base at its known position in the overhang;
(c) a particular base (selected from all possibilities) at the position of one of the random bases; and
(d) all combinations of random bases at the other positions.

Thus each family of a library differs in the identity of the base(s). All members of any one family may be fluorescently labelled so as to be detectably different from the members of the other family.

The two libraries differ in the identity of the semi-random base (b).

The restriction digest is interrogated with each library of probes in turn. For any fragment that is homospecific then the overhangs of that fragment will bind to two fragments from one of the libraries. For any fragment that is heterospecific that fragment will bind to one probe from one library and another probe from the other library. It is possible to determine from the detection signatures which probes have bound. It can then be determined for each of the overhangs which two bases are present at the position being interrogated by the base (c).

The procedure for determining the identity of the other random bases (as one of at most two possibilities) in the overhangs may be repeated using a procedure as outlined above but with the variable base at different positions. Whilst the results of this analysis will identify for any particular fragment, the two bases that are present at the respective positions being interrogated in each of the overhangs, it will not necessarily identify the overhang with which that base is associated.

In order to identify the identity of a base at a given position where the base has been determined as being one of two possibilities, libraries of probes may be used which
(i) identify whether a base at a particular position is one of T or G or one A or C;
(ii) identify whether a particular base at a particular position is one of C or G or one of A or T; or
(iii) identify whether a particular base at a particular position is one of C or G or one of A or T.

By a comparison of the results obtained with (i) and (ii), or (i) and (iii), or (ii) and (iii), or (i) and (ii) and (iii) it is possible to determine the identity of each base at a particular position.

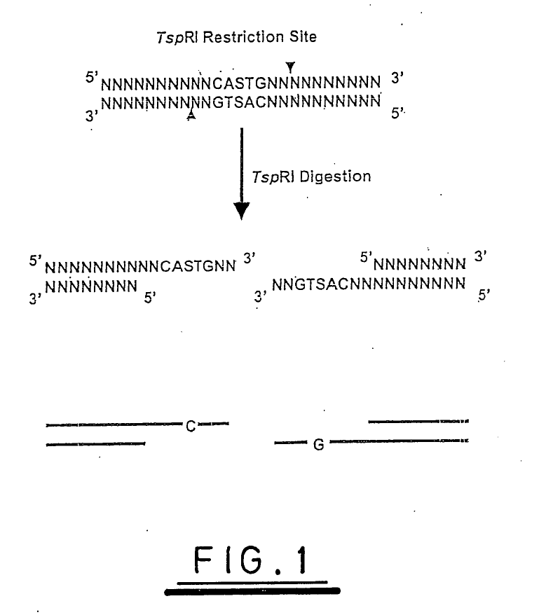

The invention will be explained with reference to the use of TspR1 as the restriction enzyme. The restriction site for TspR1 is shown in FIG. 1a for which the letters A,T,G and C have their conventional meaning for DNA, S is either G or C, and N is any one of A,T,G and C. On digestion the 3′-overhangs illustrated in FIG. 1b are produced. It will be appreciated that for one of the illustrated overhangs S will be C and correspondingly in the other overhang S will be G. For convenience the overhangs in which S═C are designated herein as sequence C-type and those in which S=G as G-type.

It will be appreciated that digestion at the restriction site illustrated in FIG. 1(a) produces two fragments, one with the C-type overhang and the other with GSG. Which fragment carried G-type and which carries C-type will depend on the orientation of the restriction site within the sequence which, under most circumstances, will be random.

It will be further appreciated that the digestion produces fragments with overhangs as described at each end. (These fragments will of course be of different lengths depending on the “spacing” between the restriction sites in the original target nucleic acid). Each such fragment will have one of three possible combinations of C-type and G-type overhangs. More particularly, one quarter of each of the fragments will (statistically) carry the G-type overhang at each end of the fragment (G-type/G-type), one quarter will carry the C-type overhang at each end of the fragment (i.e. C-type/C-type), and the remaining half will carry one of each type of overhang (i.e. C-type/G-type).

It should be further appreciated that a C-type overhang (which is of the formula NNCACTGNN) may have one of 256 sequences depending on the nucleotides N. Similarly G-type overhang can also have any one of 256 sequences.

Consider now a library of 256 primers (the “C primers”) having or incorporating a sequence of the general formula

| | |
|---|---|
| NNCACTGNN | C-type primer |

(where in the library the four Ns take all possible combination of A,C,T and G). These primers will selectively hybridise only to the G-type overhangs. Similarly a library of 256 primers (the “G-type primer”) having or incorporating the sequence

| | |
|---|---|
| NNCAGTGNN | G-type primer |

(where the four Ns have all possible combinations of A,C,G and T) will hybridise only to the C-type overhangs.

Put another way, the library of C primers will under the appropriate hybridisation conditions hybridise to all of the G-type overhangs in the restriction digest. Similarly the library of G primers will hybridise to all of the C-type overhangs in the digest.

The C-type primer and G-type primer libraries are used in an initial step of the analysis to ascertain of each fragment of the combination of overhangs (G-type or C-type) at its ends.

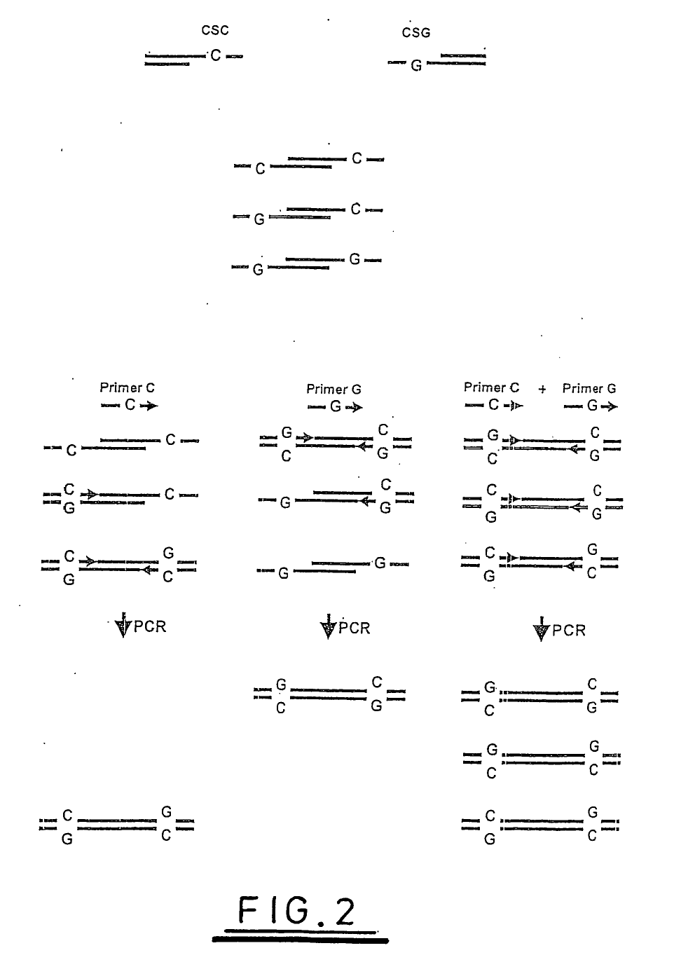

A way in which this may be effected is illustrated in FIG. 2 which at (b) illustrates the various combinations of overhangs each fragment of the digest may have. Three samples of the digest are taken. One sample is treated (under the appropriate hybridising conditions) with the C-type primer library. A further sample is treated with the G-type primer library. The remaining sample is treated with both the C-type primer and G-type primer libraries.

As shown in FIG. 2, treatment with the C-type primer library by itself results in binding to fragments with G-type overhangs only and therefore the fragment that have either the G-type/G-type or G-type/C-type combination of ends. Similarly treatment with the G-type primer library by itself results in binding to fragments having either the C-type/C-type or C-type/G-type combination of ends. Simultaneous treatment with both the C-type primer and G-type primer libraries result in binding to all fragments.

Once each sample of the digest has been treated with the appropriate primer library (or mixture thereof) the hybridised primers may be ligated and optionally treated with a DNA polymerase under reaction conditions such that the ligated target is extended at its 3′ ends to render it double stranded. The sample may also be optionally treated using a suitable exonuclease to remove sequences which are not perfectly matched. The resultant constructs are then amplified using PCR employing the same primers for amplification as we used for ligation. Thus the sample originally treated only with the C-type primer library for hybridisation/ligation is treated with the C-type primer library for amplification. As a result only the fragments with the G-type/G-type combination of ends are amplified (and not those with the SCG/C-type combination to which the C-type primers also originally amplified prior to ligation).

Similarly the sample originally treated with only the G-type primer library of hybridisation/ligation is treated with the G-type primer library for PCR amplification and gives amplified fragments having the SCS/SCS combination of overhangs.

The sample initially treated and subsequently amplified with the mixture of the C-type primer and G-type primer library will provide amplification of all fragments.

The three samples may now be subjected to size separation and detection, e.g. on an agarose gel, using known techniques. The sample treated/amplified only with the C-type primer library will give numerous bands (only one illustrated at the foot of FIG. 2) all of which will be derived from the original digestion fragments having G-type/G-type combination of each and which represents such digestion fragments of different length. Similarly the sample treated/amplified only with the G-type primer library will produce bands (only one illustrated) of digestion fragments of different length of ends. The other sample treated/amplified with the mixture if the two primer libraries generate band for all fragments (three illustrated at the bottom of FIG. 2).

By comparing the three results it is possible to assign to each fragment the combination of ends it possesses. Thus the uppermost and lowermost bands shown for the results of the mixed library treatment can be seen (from comparison with the single library treatments) to be derived from digest fragments having the C-type/C-type and G-type/G-type combinations respectively. The remaining band in the mixed library treatment must be derived from a digest fragment with the combination C-type/G-type since an equivalent band does not appears in the samples treated with a single library.

The above described procedure utilised, for the PCR reaction, the 9 base primers of the C-type primer and G-type primer libraries. It is normal to perform PCR reactions with primers longer than the TspR1 cleavage product. Should it be required, longer primers could be synthesised to carry the C-type primer or G-type primer sequence at their 3′ ends, and these could be used for the hybridisation/ligation and/or PCR amplification method described in FIG. 2.

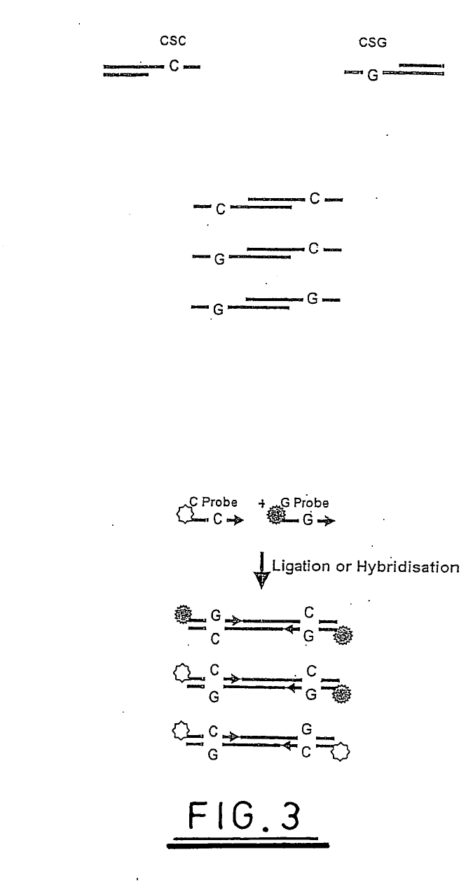

As an alternative to the method of FIG. 2, the procedure illustrated in FIG. 3 may be used for determining the combination of ends (C-type/C-type, G-type/G-type or C-type/G-type) for the fragments of the restriction digest.

In this case fluorescently labelled probes having the general formula described above are synthesised, such that the primers of the G-type primer library have a detectably different fluorescent tag from those of the C-type primer library. By hybridising the probes to the target fragments in a sequence specific manner, then separating the fragments by, for instance, agarose gel electrophoresis, the probe carried by each fragment will reflect the nature of the cleavage sequences at ends of the fragment. Whether hybridisation is sufficient to label the fragments or whether ligation is required to covalently lock the probes in position first would depend upon the nature of the separation process, if indeed a separation process is required to identify the tags carried by individual fragments. It is possible that an aggressive separation process might remove probes held only by hybridisation while ligation might prevent this. Processes known in the art for the optimisation of ligation, such as dephosphorylation of the target to prevent self-ligation of target fragments, would have obvious application in the method of the invention.

The methods outlined in FIGS. 2 and 3 obviously give some information as to the nature of the Cleavage Sequences held by individual fragments and this can be used when composing a restriction map of the DNA target. For instance as the cleavage reaction generates different cleavage sequences on each resultant fragment, it is not possible for two fragments with the same cleavage sequence (G-type or C-type) to have arisen from contiguous sequence on the original DNA double strand. It is equally apparent that the information obtained would not be sufficient to construct a map of TspRI restriction sites that also identified which fragments were originally contiguous with which others, except for simple cases involving a very restricted map.

More information can be generated by interrogating the cleaved fragments to identify further nucleotides within their cleavage sequences.

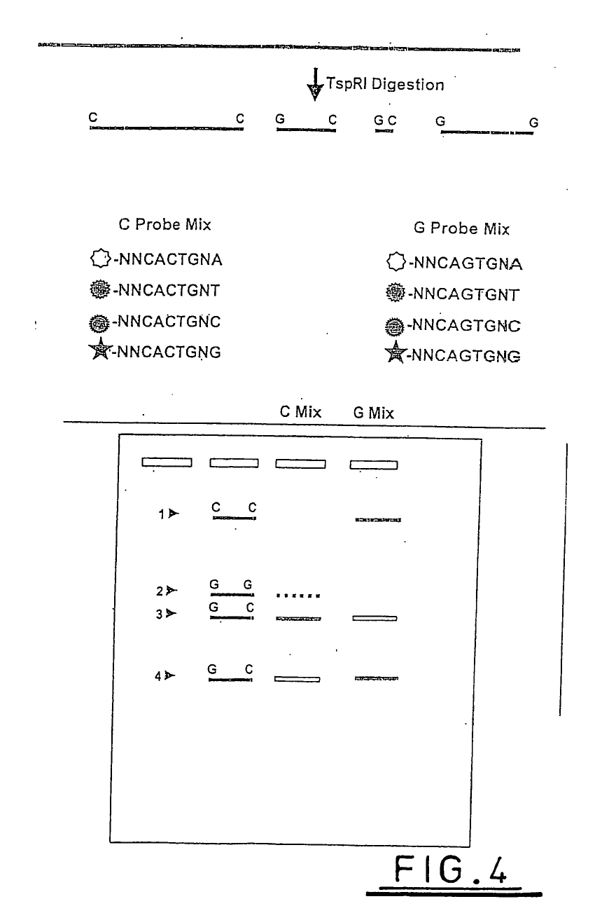

This may be done in a number of ways. One way is shown in FIG. 4 which, for the purposes of illustration, assumes that the digest produced four fragments, one was the C-type/C.-type combination of ends, one with the G-type/G-type combination and two with the C-type/G-type combination. Assume that we know, from the type of initial study shown in FIGS. 2 and 3, the nature (G-type or C-type) of the cleavage sequence but not the disposition of fragments on the original DNA double strand or the sequences of the cleavage strands. A TspR1 restriction digest is hybridised with and ligated to two mixes of probes identified in FIG. 4 as the “C Probe Mix” and the “G Probe Mix”. The C Probe Mix contains four “families” of labelled probes of the general sequence NNCACTGNX. Each family has a particular base (A, G, C or T) for X and comprises 64 probes corresponding to all possible combinations of N, each probe in any particular family having a fluorescent label which is detectably distinguishable from that used for any of the other families. Similarly the probes of the G Probe Mix are of the general formula NNCAGTGNX.

A sample of the restriction fragment digest is treated with the C Probe Mix and the probes ligated.

It will be appreciated that certain of the probes from the C Probe Mix will hybridise to G-type overhangs. The probes that will bind are those having their variant terminal base X complementary to the terminal base of G-type overhang. It is possible to determine the terminal base of the overhang from the fluorescent profile of the hybridised/ligated construct,. Thus the ligation of a restriction digest fragment having a G-type overhang and with A at the final position would be identified by the fluorescent signal associated with binding/ligation of a probe form the C Probe Mix having T as the terminal base

Similarly the digest is treated with the G Probe Mix and the probes ligated.

Following ligation the molecules are differentiated by agarose gel electrophoresis and the fluorescence profile of each band is read (FIG. 4(c)). Fragment 1 gives no fluorescence with the C Probe Mix because from the initial study its ends are known to be both of the C-type type and therefore non-complementary. With the G Probe Mix the signal is illustrated as being a composite of the signals given by the T and C associated fluorophores. This indicates that the fragment 1 has ends of the composition GNCACTGNN and ANCACTGNN. Fragment 2 is assumed to have overhangs G-type at both ends and with the C Probe Mix is assumed to give a profile showing colours equivalent to both A and C in the final variant (X) position. This shows that the two ends of the fragment 2 are composed of cleavage sequences TNCAGTGNN and GNCAGTGNN.

It will be appreciated that fragment 2 does not give any signal with the G Probe Mix.

Fragments 3 and 4 are of the mixed C-type/G-type and thus will generate a signal following ligation with both the C Probe and G Probe Mix. Also, the fluorophore profile associated with ligation to each mix unequivocally assigns the associated base to one end or the other. Fragment 3 gives the signal associated with C from the C Mix and A from the G Mix. Thus fragment 3 has ends with the sequence GNCAGTGNN and TNCACTGNN, while fragment 4 (A signal from the C Probe Mix and T signal from the G Probe Mix) has sequences TNCAGTGNN and ANCACTGNN.

By using similar pairs of fluorescently labelled C Probe and G Probe Mixes wherein the fluorescent tag specific base (X) is positioned at the other variant positions (i.e. NNCA(C/G)TGXN, NXCA(C/G)NN and XNCA(C/G)NN) information can be gathered regarding the disposition of bases within the cleavage sequences of the fragments.

In the case of heterospecific cleavage sites (i.e. fragments containing one C-type and one G-type overhang) this allocation of bases can be unequivocal because each C Mix or G Mix ligation will address only one cleavage site on the fragment. As a result, the above procedure allows the 9 base cleavage sequence at the end of each heterospecific fragment (which should constitute approximately 50% of the fragment population) to be fully identified.

In the case of homospecific cleavage sites (i.e. fragments containing C-type/C-type or G-type/G-type overhangs) the information is less clear cut as these cleavage sites are not addressed individually by this method. While the pair of nucleotides present on homospecific fragments cleavage sites can be determined for each variant position, which base is at which particular end cannot be established. Put another way, whilst the C Probe and G Probe Mixes illustrated in FIG. 4 will identify the bases present at each end of the homospecific fragments, the subsequent C Probe Mixes (i.e. NNCA(C/G)TGXN, NXCA(C/G)NN and XNCA(C/G)NN) will identify, for each X position, two bases but will not discriminate as to which end of the fragment each base is located. It is possible, particularly in the case of simple maps with a limited number of digest fragments that the data could be deconvoluted by logical deduction from the fact that each cleavage yields two cleavage sites which must be complementary and while one may partition onto a homospecific fragment there is an even chance that its complement is on a heterospecific partner, which will allow its sequence to be fully elucidated. Comparison of those known sequences that do not have a fully sequenced complement with the pool of homospecific semi-defined sequences might allow assignment of complements if no other potential complement exists.

As an example if the signals produced for each variant position in a C-type homospecific fragment were (C and T, G and C, T alone, A and T) then both cleavage sites are of the following formula;
(T or C)(G or C)CACTGT(A or T)

The emboldened T can be assigned unequivocally, as this was the only signal produced for this position.

In order to maximise the information available in the restriction map it is necessary unequivocally to ascribe cleavage sequences to the homospecific fragments above.

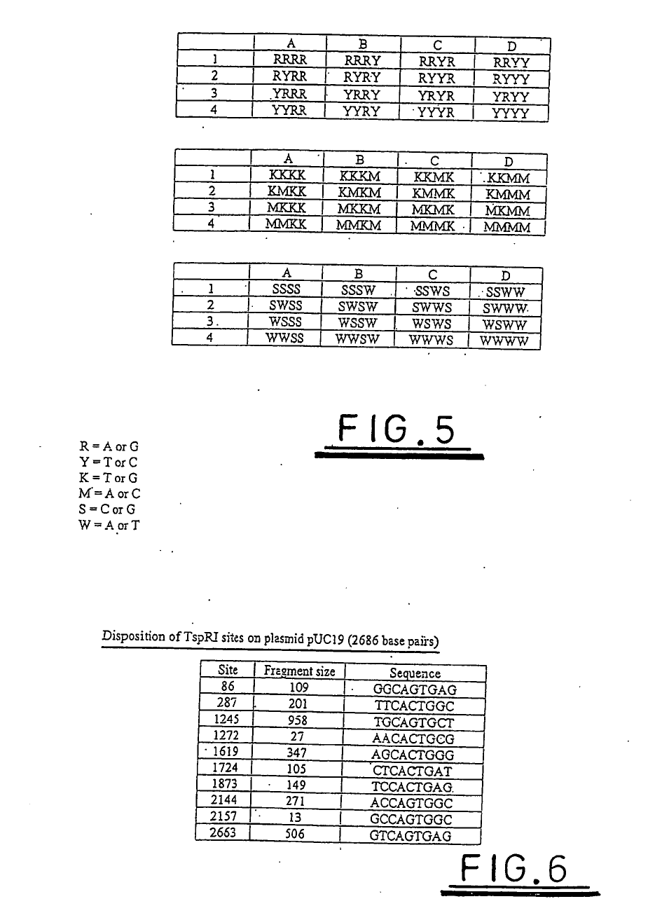

This can be achieved in a number of ways, one of which is based on pools of probes which will be described with reference to FIG. 5. The pools comprise degenerate probes created containing all combinations of cleavage sequence.

These will have the general formula NNCASTGNN where S═C or G so that the pool will hybridise with either C-type or G-type sequences. For present purposes it is convenient to refer to the four nucleotides (NNNN) at the ends of the fragment while ignoring the common (CASTG) sequence in the middle of the sequence.

The pools are synthesised separately in such a manner that each carries one of four distinguishable fluorescent (or other) markers (1 to 4 in the tables of FIG. 5).

Three separate pools are made. Each pool contains a mixture of C-Probes and G-Probes. Thus, for example, the RRRR probes of cell A1 of the first Table are made up of 32 probes, i.e. 16 C-Probes and 16 G-Probes including all possible combinations of R. In the first pool (i.e. the first Table of FIG. 5) the probes are split into all sixteen combinations of purine(R) or pyrimidine (Y) residues in the four N positions. These are synthesised as in FIG. 5, with four combinations synthesised with each of four fluorophores. This allows four differently marked combinations to be used in each of four ligation reactions. Ligation reactions are then performed, as described elsewhere, with probes grouped as in columns A to D of the tables, so that each reaction carries four degenerate probes each differently labelled. The products of this reaction may then be detected in a manner somewhat similar to that described for FIG. 4.

In the second pool a similar set of combinations is synthesised to contain all combinations of T or G (K), or A or C (M) in the four N positions.

The third pool (which may or may not be required for the analysis of a particular sequence—see below) contains all combinations of C or G (S) and A or T (W) in the four N positions. It is possible that, say, only the first and third pools may be sufficient so that the second pool is not required? Similarly the second and third pools may be sufficient so the first pool is not required.

By performing the equivalent ligation reactions the disposition of K/M and S/W residues may be determined within each cleavage site. Each fragment will give two RY combinations and two KM combinations (one for each end). These may be compared to the table below to give two possible cleavage fragment combinations. The data generated by the first part of the analysis (i.e. the pair-wise combinations of residue at each base position) can be used to discriminate these possibilities.

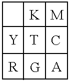

For example, take a hypothetical homo-specific fragment whose ends have the NNNN combinations TGAT and TGGA. The previous round of analyses would determine the central ambiguous C or G for the ends and that the NNNN residues were T, G, A or G, and A or T.

With reference to the tables below, the present phase of analysis would identity the variable bases on the fragment overhangs as belonging to specific subsets (YRRY,YRRR and KKMK,KKKM) on the basis that they hybridised to the complements of these sequences in the families of primers in the Y/R and K/M sets. This equates to ends of (TGAT and TGGA) or (TGGC and TGAG). It can be seen that the second combination of sequences does not equate to the pair-wise bases identified in the first analysis while the first pair of final sequences agree fully with this analysis and are those chosen for the example.

It can be shown however, that for any NNNN combination at one end of a fragment there are fifty of the 256 possible NN combinations at the other end of the fragment that cannot be unequivocally distinguished on the basis of first analysis together with RY and KM analysis. It is for these cases that the third pool analysis (S/W) is performed as this can unequivocally distinguish the 50 residue pairs |(for each NNN combination) which cannot be otherwise distinguished. This is outlined more fully in the Example below.

From the above the following particularly advantageous properties of the enzyme TspRI make it the most preferred for use in this application:
i) the restriction site is 9 base pairs in length, which is useful in extending from a nononucleotide primer for certain applications. It may be possible to directly PCR products from 9-mer primers or longer primers with the specific 9-mer sequences at their 3′ ends.
ii) The recognition sequence is CASTG and contains four whole nucleotides and one half nucleotide (S denotes that C or G at position 5 will cut while A or T will not) and would thus arise, statistically, once every 512 bases in a sequence. This is ideal size for such applications as sequencing.
iii) Following cleavage a 9 base overhang is left and of these nine bases 4 are completely random and one is half-random (C or G in position 5). Thus there is a potential to discriminate 512 different sequences at the cleavage site.

The above described techniques employ various hybridisation reactions. It is known in the art that under a given set of reactant conditions (of, for instance, salt concentration) the hybridisation (and subsequent ligation) of a probe to its target is temperature dependent. Control of the temperature at which hybridisation occurs (the so-called stringency of hybridisation) can affect the length and degree of sequence complementarily necessary to effect efficient hybridisation. It is also known in the art that the binding of guanine to cytosine is effected by two hydrogen bonds while that of thymine and adenine is effected by a single hydrogen bond. As a result, the stability of a fully complementary sequence of a fixed length will be dependent upon the ratio of G/C:A/T base pairings (i.e. higher ratios having a higher dissociation or melting temperature).

It is possible that this sequence dependence of the hybridisation reaction may adversely effect the fidelity of detection of variant sites at the end of the probe furthest from the site of ligation. Under certain conditions of reactant composition and temperature non-complementary sequences may hybridise, generating a false signal. It is possible to eliminate the false signal generated by such mismatch ligation by treating the ligated mix with an enzyme which will specifically degrade such mismatch regions while not effecting fully complementary matches. This enzymatic digestion would remove the end of the probe carrying the fluorophore such that, while the mismatched species would still be present in the fragment mix on a subsequent agarose gel it would not generate a signal and therefore would not interfere with sequence identification. Suitable enzyme would include Mung Bean Nuclease and T7 endonuclease (New England Biolabs catalogue 2000-2001, www.neb.com). The method of the invention optionally includes the removal of mismatched fragment signal by such enzymatic digestion.

It is also known that oligonucleotide analogues exist, such as Peptide Nucleic Acid (PNA) or Locked Nucleic Acid (LNA) which are much less dependant on salt concentration for binding and which bind much more strongly to DNA than a second strand of DNA. It could be envisioned that the methods of the invention would be amenable to the use of such analogues, with or without ligation.

As will be obvious to anyone skilled in the art, following the disclosures of the invention will be useful for a variety of purposes beyond simply mapping a DNA double strand sequence. For instance having knowledge of the sequence and disposition of 9 base sequences within a fragment or region of interest would be extremely useful in informing sequencing strategies and may also be used with suitable linker or vector molecules for the directed sub-cloning of the region (i.e. TspRI fragments or partial digests containing a number of contiguous TspRI fragments may be generated by partial digestion and subcloning or by addition of linkers and sub-cloning or by PCR amplification. Also, knowledge of such disposition within a number of fragments would obviously be useful in combining the information to obtain a map of a larger region than any individual fragment might hold.

The invention is illustrated by the following non-limiting Examples.

### EXAMPLES 1

### Theoretical

A hypothetical analysis is illustrated with reference to the explanation supplied above and to FIGS. 6 to 10. Plasmid pUC19 is a double stranded circle of DNA, 2686 base pairs in length, whose sequence is fully known and, therefore, whose digestion with TspRI can be fully predicted.

FIG. 6 shows the sequence (upper strand only) at the expected restriction digestion sites for TspRI.

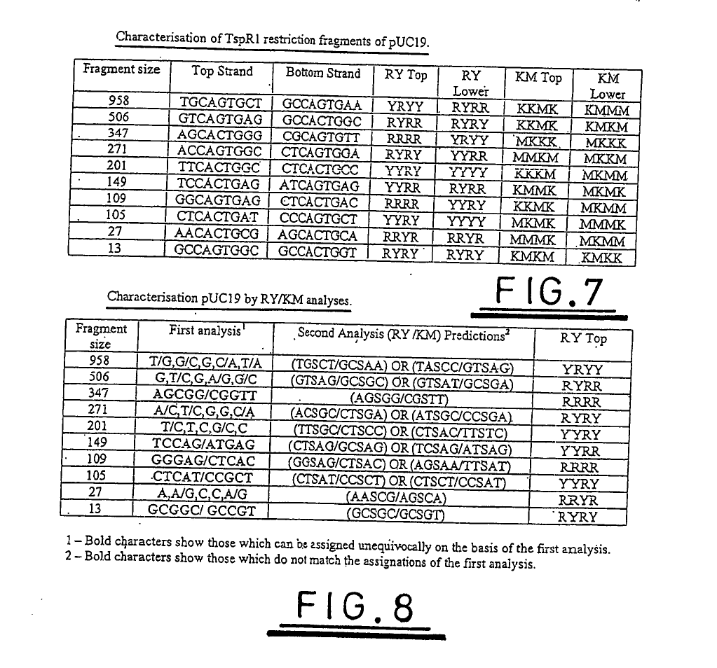

FIG. 7 shows the two overhanging 9 base sequences which would be present on each fragment generated by digestion with the enzyme and the predicted sequences that each would generate with the YR and KM pools of labelled oligonucleotides outlined above.

The disposition of these fragments following (hypothetical) agarose gel electrophoresis of is shown in FIG. 10. In practice it might be preferable to perform SW analysis in concert with the KM/RY analyses shown, however this is omitted from the figure to simplify the diagram in order to aid its understanding.

FIG. 8 demonstrates how the information from the first analysis and RY/KM analyses may be combined to ascribe sequences to the TspRI digestion sites in most cases. From this figure it can be seen that two of the fragments (105 and 271 base pairs) give unequivocal results using KM/RY analysis alone. One of these (105 bp) can be fully ascribed on the basis of the first analysis, as it is a hetero-specific fragment of the type described above.

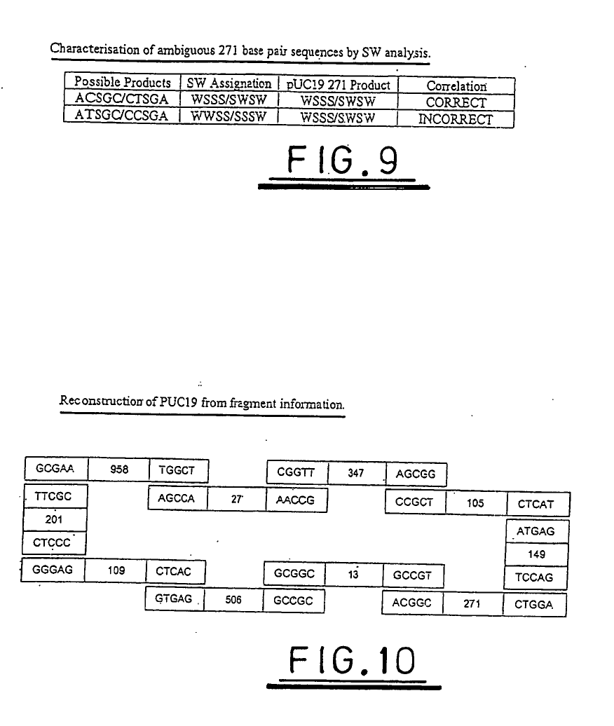

FIG. 9 shows how the second unassigned fragment (271 bp) may be assigned on the basis of SW analysis, in combination with the other analyses performed.

FIG. 10 demonstrates how the disposition of TspRI sites in pC19 might be used to map the plasmid. Comparison of this figure with FIG. 6 shows that an accurate map has been constructed.

### EXAMPLE 2

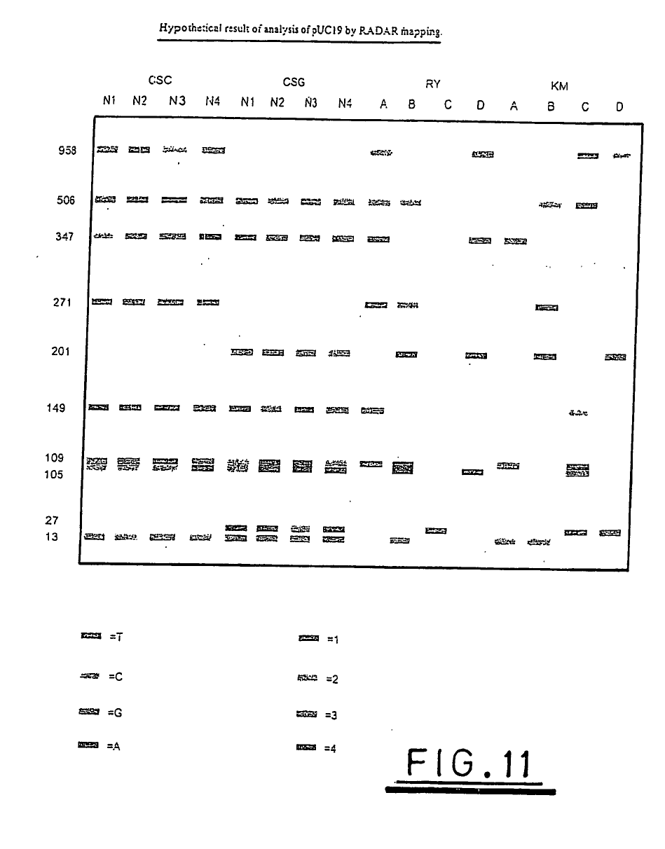

The following experiment demonstrates the ligation of fluorescently tagged probes to TspRI restriction digest fragments of plasmid pUC19 and is illustrated with reference to the explanation supplied above and to FIGS. 6 to 12.

The following 9 base probes were synthesised

5′ JOE-NNCACTGNA-3′
5′ FAM-NNCACTGNT-3′
5′ ROX-NNCACTGNG-3′
5′ TAMRA-NNCACTGNC-3′

JOE, FAM, ROX and TAMRA are fluorescent dye molecules known in the art, which can be distinguished on the basis of the wavelength at which fluorescent light is emitted from them. A probe mix was formulated containing 20 pmoles/μL of each probe. This is referred to as probe mix.

Plasmid pUC19 DNA (10 μL of 1 mg/μL solution.—New England Biolabs (NEB), catalogue number #304-1S) was digested with TspRI at 65° for 100 minutes in the following reaction mix;

| | |
|---|---|
| PUC19 (1 mg/mL) | 10 μL |
| NEB Buffer 4 (10x concentrate) | 10 μL |
| Bovine Serum albumen (20 mg/mL) | 5 μL |
| Molecular Biology grade water (MB water) | 70 μL |

This was thoroughly mixed and then 5 μL of TspRI (NEB-5 U/μL) mixed in and incubated as above. Following incubation the DNA was recovered by ethanol precipitation and resuspended in 100 μL of MB water.

The probe mix was then hybridised to the fragments and ligated into position using T4 DNA ligase (400 U/μL, NEB #202S), with appropriate control reactions, in the following four reaction mixes (A to D);

| | A | B | C | D |
|---|---|---|---|---|
| TspRI digested pUC19 DNA | 2 μL | 2 μL | 2 μL | 2 μL |
| Probe mix | 0 μL | 0 μL | 10 μL | 10 μL |
| T4 ligase buffer (NEB) | 2.5 μL | 2.5 μL | 2.5 μL | 2.5 μL |
| MB water | 20.5 μL | 19.5 μL | 6.5 μL | 1.5 μL |
| The reactions were thoroughly | | | | |
| mixed and then; | | | | |
| T4 DNA ligase | 0 μL | 1 μL | 1 μL | 1 μL |

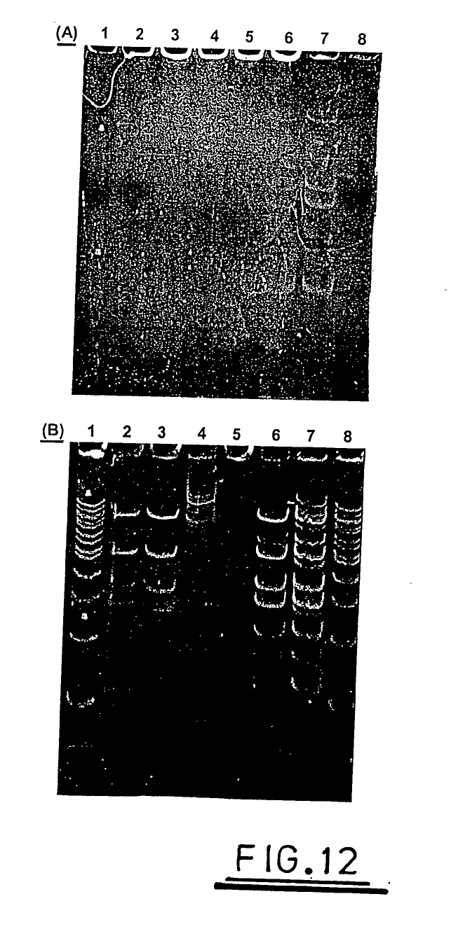

The ligation was incubated at 30° C. for 90 minutes then 5 μL of 10× concentrated E. coli exonuclease I buffer (NEB, B0293S), 19 μL of MB water and, after mixing, 1 μL of E. coli exonuclease I (20 U/μL, NEB M0293S) were added and incubation continued at 37° C. for 30 min. The exonuclease treatment is performed to degrade unincorporated labelled probe which can otherwise interfere with gel interpretation. After this the samples were ethanol precipitated and resuspended in 5 μL of MB water and then 1 μL of 50% (w.v) glycerol added (without gel running dye which might obscure results). The samples were then subject to 10% non-denaturing polyacrylamide gel electrophoresis. Controls included on the gel were 2 μL of unhybridised TspRI cut pUC19 and 100 bp ladder DNA standards (Promega). The gel was photographed under UV illumination to determine fluorescent bands and then the gel was immersed in 0.5 μg/mL ethidium bromide solution for 15 minutes and rephotographed to determine the positions of all visible DNA strands. The photographs in FIG. 12 show the results of this experiment with photograph (A) taken before ethidium bromide staining and photograph (B) taken afterwards. In both photographs the lane notation is as follows;
Lane 1—100 bp DNA fragment ladder
Lane 2—unhybridised TspRI cut pUC19
Lane 3—reaction A
Lane 4—reaction B
Lane 5—empty
Lane 6—reaction C
Lane 7—reaction D
Lane 8—100 bp DNA fragment ladder

From the photographs it is clear that fluorescence is apparent in lanes 6 and 7 of gel A (i.e. before general DNA staining) and this can be presumed to arise through ligation of fluorescent probes to these fragments. No sequence identity can be obtained from this photograph as it holds no colour data.

1-22. (canceled)

23. A method of analysing a nucleic acid sequence comprising
(A) digesting the sequence with a restriction enzyme which cleaves the nucleic acid sequence to produce a digest including a fragment mixture having known bases at known positions n random bases at known positions, and a semi-random base at a known position; and
(B) analysing the fragment mixture obtained from (A) to determine the relative size of the fragments and the sequences of their ends, wherein step (B) itself comprises the steps of:
(i) analysing the fragment mixture to determine the identities of the semi-random bases in each overhang at each end of the fragment;
(ii) analysing the fragment mixture to identify each random base in the two overhangs of a fragment so as uniquely to identify that base or identify it as being one of two possibilities; and
(iii) identifying the identity of a random base at a given position where that base has been determined as being one of two possibilities.

24. A method as claimed in claim 23 wherein the sequence data derived from step B(iii) is used to order the fragments relative to each other to generate partial or complete restriction maps of said nucleic acid sequence.

25. A method as claimed in claim 23 wherein step B(i) is effected using first and second libraries of probes, each library being comprised of probes having a sequence for potential hybridisation to the overhangs,
the probes of the first library having, at a position corresponding to the semi-random base, one of the two possibilities therefor,
the probes of the second library having, at a position corresponding to the semi-random base, the other of the two possibilities therefor,
said two libraries being used separately of each other and also together to interrogate separate samples of the digest, and
analysing the fragment mixture to identify the relative sizes of the fragments and also for each size fragment whether it has a different semi-random base in its two overhangs or has the same semi-random bases in its two overhangs.

26. A method as claimed in claim 23 wherein step B(ii) is effected by analysing the digest from (A) in turn with third and fourth primer libraries each having n “families” of primers with each “family” of a particular library including probes with sequences comprising:
(a) bases complementary to said known bases of the overhang at their known positions;
(b) a semi-random base at its known position in the overhang;
(c) a particular base (selected from all possibilities) at the position of one of the random bases; and
(d) all combinations of random bases at the other positions,
the third and fourth libraries differing in the identity of the semi-random base (b) and the “families” of each library being fluorescently labelled so as to be detectably different from the members of the other families.

27. A method as claimed in claim 23 wherein step B(iii) is effected by using libraries of probes which
(I) identify whether a base at a particular position is one of T or G or one A or C;
(II) identify whether a particular base at a particular position is one of C or G or one of A or T; or
(III) identify whether a particular base at a particular position is one of C or G or one of A or T, and
comparing the results obtained with (I) and (II), or (I) and (III), or (II) and (III), or (I) and (II) and (III) to determine the identity of each base at a particular position.

28. A method as claimed in claim 23 wherein the restriction enzyme produces overhangs of 6 or more bases.

29. A method as claimed in claim 23 wherein at least three of the bases in the overhang are random.

30. A method as claimed in claim 26 wherein the enzyme is TspRI.

31. A method as claimed in claim 30 wherein step B(i) is effected a “C-primer” library having or incorporating a sequence of the general formula NNCACTGNN and a “G-primer” library having or incorporating the sequence NNCAGTGNN, wherein in each library the four Ns take all possible combinations of A, C, T and G, the method comprising treating a first sample of the digest obtained from Step (A) with the “C primer” library, a second sample of the digest obtained from Step (A) with the “G-primer” library, and a third sample of the digest obtained from (A) with both the “C-primer” and “G-primer” libraries, and subjecting the treated digests to size separation and detection to determine for each fragment the combination of semi-random bases possessed by its ends.

32. A method as claimed in claim 30 wherein for step B(ii) separate samples of the digest are probed separately with families of probes of the formula

| | | | |
|---|---|---|---|
| (w) | NNCACTGNX | and | NNCAGTGNX |
| (x) | NNCACTGXN | and | NNCAGTGXN |
| (y) | NXCACTGNN | and | NXCAGTGNN |
| (z) | XNCACTGNN | and | XNCAGTGNN |

wherein for any one family, X comprises all possible bases and N represents all combinations of random bases at other positions and probes with the same X are labelled identically but distinguishably from probes with different X.

33. A probe library consisting of probes having or containing the sequence NNCACTGNN where N is A, C, T or G and the library comprises probes in which each N is all four possibilities.

34. A library as claimed in claim 33 wherein the probes are labelled.

35. A probe library consisting of probes having or containing the sequence NNCAGTGNN where N is A, C, T or G and the library comprises probes in which each N is all four possibilities.

36. A library as claimed in claim 35 wherein the probes are labelled.

37. A probe library consisting of probes having or containing the sequences NNCACTGNN where N is A, C, T or G in which each N is all four possibilities and having or containing the sequences NNCAGTGNN where N is A, C, T or G in which each N is all four possibilities.

38. A probe library consisting of probes having or containing sequences comprised of the following families (i)-(iv):

| | |
|---|---|
| (i) | NNCACTGNA |
| (ii) | NNCACTGNT |
| (iii) | NNCACTGNC |
| (iv) | NNCACTGNG |

wherein N is A, C, T or G, each family (i)-(iv) comprises sequences corresponding to all values of N and the members of any one family are labelled so as to be detectably different from the other families.

39. A probe library consisting of probes having or containing sequences comprised of the following families (i)-(iv):

| | |
|---|---|
| (i) | NNCACTGAN |
| (ii) | NNCACTGTN |
| (iii) | NNCACTGCN |
| (iv) | NNCACTGGN |

wherein N is A, C, T or G, each family (i)-(iv) comprises sequences corresponding to all values of N and the members of any one family are labelled so as to be detectably different from the other families.

40. A probe library consisting of probes having or containing sequences comprised of the following families (i)-(iv):

| | |
|---|---|
| (i) | NACACTGNN |
| (ii) | NTCACTGNN |
| (iii) | NCCACTGNN |
| (iv) | NGCACTGNN |

wherein N is A, C, T or G, each family (i)-(iv) comprises sequences corresponding to all values of N and the members of any one family are labelled so as to be detectably different from the other families.

41. A probe library consisting of probes having or containing sequences comprised of the following families (i)-(iv):

| | |
|---|---|
| (i) | ANCACTGNN |
| (ii) | TNCACTGNN |
| (iii) | CNCACTGNN |
| (iv) | GNCACTGNN |

wherein N is A, C, T or G, each family (i)-(iv) comprises sequences corresponding to all values of N and the members of any one family are labelled so as to be detectably different from the other families.

42. A probe library consisting of probes having or containing sequences comprised of the following families (i)-(iv):

| | |
|---|---|
| (i) | NNCAGTGNA |
| (ii) | NNCAGTGNT |
| (iii) | NNCAGTGNC |
| (iv) | NNCAGTGNG |

wherein N is C, A, G or T, each family (i)-(iv) comprises sequences corresponding to all values of N and the members of any one family are labelled so as to be detectably different from the other families.

43. A probe library consisting of probes having or containing sequences comprised of the following families (i)-(iv):

| | |
|---|---|
| (i) | NNCAGTGAN |
| (ii) | NNCAGTGTN |
| (iii) | NNCAGTGCN |
| (iv) | NNCAGTGGN |

wherein N is C, A, G or T, each family (i)-(iv) comprises sequences corresponding to all values of N and the members of any one family are labelled so as to be detectably different from the other families.

44. A probe library of consisting probes having or containing sequences comprised of the following families (i)-(iv):

| | |
|---|---|
| (i) | NACAGTGNN |
| (ii) | NTCAGTGNN |
| (iii) | NCCAGTGNN |
| (iv) | NGCAGTGNN |

wherein N is C, A, G or T, each family (i)-(iv) comprises sequences corresponding to all values of N and the members of any one family are labelled so as to be detectably different from the other families.

45. A probe library consisting of probes having or containing sequences comprised of the following families (i)-(iv):

| | |
|---|---|
| (i) | ANCAGTGNN |
| (ii) | TNCAGTGNN |
| (iii) | CNCAGTGNN |
| (iv) | GNCAGTGNN |

wherein N is C, A, G or T, each family (i)-(iv) comprises sequences corresponding to all values of N and the members of any one family are labelled so as to be detectably different from the other families.

46. A probe library consisting of probes comprising or containing the following combination of sequences

| | | | |
|---|---|---|---|
| A | B | C | D |
| | | | | |
|---|---|---|---|---|
| 1 | RRCASTGRR | RRCASTGRY | RRCASTGYR | RRCASTGYY |
| 2 | RYCASTGRR | RYCASTGRY | RYCASTGYR | RYCASTGYY |
| 3 | YRCASTGRR | YRCASTGRY | YRCASTGYR | YRCASTGYY |
| 4 | YYCASTGRR | YYCASTGRY | YYCASTGYR | YYCASTGYY |

where R is A or G, Y is T or C and S is C or G, the sequences comprising all possible combinations of R, Y and S.

47. A probe library consisting of probes comprising or containing the following combination of sequences

| | | | |
|---|---|---|---|
| A | B | C | D |
| | | | | |
|---|---|---|---|---|
| 1 | KKCASTGKK | KKCASTGKM | KKCASTGMK | KKCASTGMM |
| 2 | KMCASTGKK | KMCASTGKM | KMCASTGMK | KMCASTGMM |
| 3 | MXCASTGKK | MKCASTGKM | MKCASTGMK | MKCASTGMM |
| 4 | MMCASTGKK | MMCASTGKM | MMCASTGMK | MMCASTGMM |

K is T or G, M is A or C and S is C or G, the sequences comprising all possible combinations of K, M and S.

48. A probe library consisting of probes comprising or containing the following combination of sequences

| | | | |
|---|---|---|---|
| A | B | C | D |
| | | | | |
|---|---|---|---|---|
| 1 | SSCASTGSS | SSCASTGSW | SSCASTGWS | SSCASTGWW |
| 2 | SWCASTGSS | SWCASTGSW | SWCASTGWS | SWCASTGWW |
| 3 | WSCASTGSS | WSCASTGSW | WSCASTGWS | WSCASTGWW |
| 4 | WWCASTGSS | WWCASTGSW | WWCASTGWS | WWCASTGWW |

where S is C or G and W is A or T the sequences comprising all possible combinations of K, M and S.
